(19) United States
(12) Patent Application Publication
(10) Pub. No.: US 2004/0082078 A1
(43) Pub. Date: Apr. 29, 2004

(54) TEST PEN

(76) Inventors: Hsiu-Hua Lin, Taipei City (TW); Hsiao-Wei Tsai, Peng-Hu Hsien (TW); Chih-Ming Lo, Taipei Hsien (TW)

Correspondence Address: NAIPO (NORTH AMERICA INTERNATIONAL PATENT OFFICE), P.O. BOX 506, MERRIFIELD, VA, 22116, US

(21) Appl. No.: 10/249,590

(22) Filed: Apr. 22, 2003

(30) Foreign Application Priority Data
Oct. 24, 2002 (TW) 091124835

(51) Int. Cl. G01N 33/543

(52) U.S. Cl. 436/518

(57) ABSTRACT

A test pen includes an integral hollow pen barrel. The hollow pen barrel is divided into four sections in order being a dip section, a testing and observation section, a drying section, and a sealed rear section. The dip section has a front opening allowing a slender water-absorbing member to pass therethrough. An air balance hole is provided on the dip section of the pen barrel to facilitate liquid siphoning. A pen cap for capping the dip section has a flexible inner fringe for keeping the dip section airtight. A rear plug is provided for sealing the rear section. A testing member is installed in the testing and observation section. The testing member includes a support card, a solid carrier, a conjugate release pad partially overlapping the front end of the solid carrier, an absorbing pad partially overlapping the rear end of the solid carrier, and a cover tape.

### BACKGROUND OF INVENTION

1. Field of the Invention

The present invention relates to a rapid test pen, and more specifically, to a rapid pregnancy test pen.

2. Description of the Prior Art

Pregnancy test instruments on the market are typically sold in card form packaged in a foil wrapper or other kind of wrapping paper. A user has to disassemble the package, take out the pregnancy test card, collect her urine sample and drop the sample onto a specific testing area. Because of the complex procedure and extra instruments needed, like a cup for collecting urine, conventional pregnancy test instruments are inconvenient. Besides, a prior art pregnancy test card must be used immediately after opening the package or the test result will be altered by moisture in the air. In addition, the prior art pregnancy test card is not readily portable and not easy to keep privacy.

Currently, there is no highly portable pregnancy test instrument on the market that can be used a substantial amount of time after opening the package. Consumers usually keep a pregnancy test product in a pocket after purchase to avoid an embarrassing situation, which sometimes may result in losing the pregnancy test product. Accordingly, prior art pregnancy test instruments have many defects for customers to keep the test result in longer situation when they want to collect its result.

### Summary of Invention

It is therefore a primary objective of the claimed invention to provide a modified, easy to use, and portable test pen specifically having the function of a pregnancy test for women.

According to the preferred embodiment of the claimed invention, a test pen is provided with an integral hollow pen barrel, which is divided into four sections in order being a dip section, a testing and observation section, a drying section, and a sealed rear section. The dip section has a front opening with a diameter allowing a slender water-absorbing member to pass therethrough. A pen cap including a hollow main body and an extending clipping member is provided. The hollow main body, which is sleeved on the dip section, has a flexible inner fringe for keeping the dip section in an airtight status. The extending clipping memberis provided to keep the dip section in an airtight status and clip the test pen to a breast pocket of a user to prevent loss. A rear plug is provided for sealing the rear section. A testing member composed of a support card, a solid carrier, a conjugate release pad partially overlapping the front end of the solid carrier, an absorbing pad partially overlapping the rear end of the solid carrier, and a cover tape is provided. The hollow pen barrel further includes four interior slender protrusions for fixing the testing member. In other embodiments, the testing member could be replaced with other testing agents, like agents for sexually transmitted diseases or drug abuse.

These and other objectives of the claimed invention will no doubt become obvious to those of ordinary skill in the art after reading the following detailed description of the preferred embodiment, which is illustrated in the various figures and drawings.

### BRIEF DESCRIPTION OF DRAWINGS

FIG. 1 is an exploded perspective diagram of a test pen according to the present invention.

FIG. 2 is an exploded perspective diagram of components of the test pen of FIG. 1.

FIG. 3 is a perspective diagram of the components of the test pen of FIG. 2 assembled.

### DETAILED DESCRIPTION

The present invention provides a pen-like structure test instrument for performing a pregnancy test or other similar test. The preferred embodiment of the present invention is described in the context of a pregnancy test as follows.

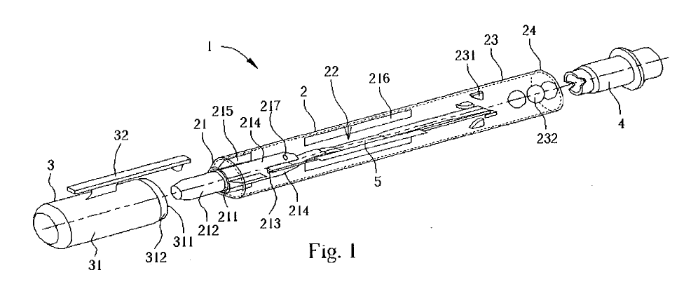

Please refer to FIG. 1, an exploded perspective diagram of a test pen 1 of the present invention. The test pen 1 comprises a transparent integral hollow pen barrel 2, a pen cap 3, a rear plug 4, and a testing member 5. The monolithic integral hollow pen barrel 2 could be made of transparent plastic. The pen barrel includes a dip section 21, a testing and observation section 22, a drying section 23, and a sealed rear section 24. As illustrated, the dip section 21 is tapered to a front opening 211 having a diameter allowing a slender water-absorbing member 212 to pass therethrough. The slender water-absorbing member 212 is fixed tightly into the opening 211 by six slender protrusions 21 5. According to the preferred embodiment of the present invention, the water-absorbing member 212 could be made of glass fiber, polyester fiber, cellulose, or other similar highly water-absorbing material. The water-absorbing member 212 is provided with a slot 213 in the rear and a tapered front part, which is similar to a pen nib of a typical marker. According to the present invention, the rear part of the water-absorbing member 212 could be cut apart to form two clips 214 and the slot 213. The clips 214 are provided to clip the testing member 5.

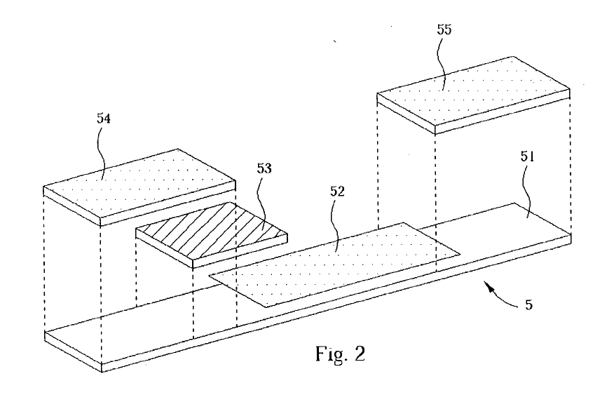

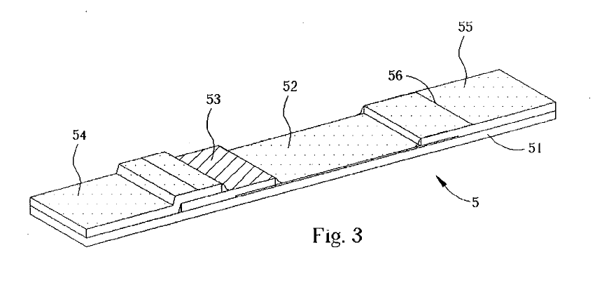

Please refer to FIG. 2 and FIG. 3. FIG. 2 is an exploded perspective diagram of the testing member 5 of the present invention, and FIG. 3 is an assembled perspective diagram of the testing member 5 of the present invention. According to the preferred embodiment of the present invention, the testing member 5 comprises a support card 51, a solid carrier 52, a conjugate release pad 53 partially overlapping the front end of the solid carrier 52, and a cover tape 56. The support card 51 is better provided in plastic in order to support the solid carrier 52, the conjugate release pad 53, a specimen-absorbing pad 54, and a rear absorbing pad 55. The solid carrier 52 is provided as a specific rectangular nitrocellulose material, such as PuraBind™, that is conducive to fluid movement and provides antigen-antibody bonding.

The conjugate release pad 53 is made of a low-density polyester material loaded with tracer, such as colloidal gold and latex having the property of being rapidly permeable by testing specimen and immediately and evenly releasing a loaded tracer after being loaded with the specimen. The rear absorbing pad 55 is a highly liquid-absorbent material, such as a blotting paper, to absorb and store the testing specimen and prevent its leakage. The specimen-absorbing pad 54 is provided overlapping the front part of the test member 5 and is made of glass fiber or cellulose fiber. The two clips 214 on the rear part of above-mentioned water-absorbing member 212 are provided to clip the specimen-absorbing pad 54 tightly. Additionally, those with ordinary skill in the art could remove the specimen-absorbing pad 54 and clip the above-mentioned water-absorbing member 212 onto the conjugate release pad 53 using the clips 214. In addition, in order to improve capillarity action by airflow, a small opening 217 normally covered by the pen cap 3 is provided an airtight situation near the front opening 211 of the integral hollow pen barrel 2.

In order to improve test efficiency of the testing member 5, the solid carrier 52, conjugate release pad 53, specimen-absorbing pad 54, and rear absorbing pad 54 should overlap partially but not be on the same horizontal plane.

Please refer to FIG. 1. The testing and observation section 22 of the integral hollow pen barrel 2 is provided over the solid carrier 52 allowing a user to observe testing results. A result could be observed in an area divided by a line in the testing and observation section 22 of a transparent integral hollow pen barrel 2, or in the transparent testing and observation section 22 of an opaque integral hollow pen barrel 2. Additionally, four slender protrusions 216 are provided to fix the upper side of the testing member 5 tightly in the middle part of the testing and observation section 22. Accordingly, testing and control lines can be observed on the solid carrier 52, which is well known to those skilled in the art.

The drying section 23 of the integral hollow pen barrel 2 loaded with at least one drying agent 232 is partitioned from the remaining part of the integral hollow pen barrel 2 by an extending fastener 231, wherein the fastener 231 is an integral part of the integral hollow pen barrel 2 provided at the end of the testing and observation section 22 to separate the drying agents 232 from the testing member 5. The size and number of fasteners 231 depends on not obstructing the assembly of the testing member 5. An opening 241 is provided in the sealed rear section 24 of the integral hollow pen barrel 2 to accept the rear plug 4 tightly.

The rear plug 4 can be made of a soft or stiff plastic material to prevent moisture from contacting the testing member 5 after the rear plug 4 is plugged into the sealed rear section 24. The pen cap 3 is provided with a hollow main body 31 and an extending clipping member 32. An opening 311 is provided in the hollow main body 31 to cap the dip section 21 of the integral hollow pen barrel 2. An inner elastic edge 312 is provided around the opening 311 to keep the integral hollow pen barrel 2 airtight by tightly fitting over the pen barrel 2. The extending clipping member 32 of the pen cap is provided to clip the pregnancy test pen on a breast pocket to avoid losing it.

In contrast to the prior art, the present invention pregnancy test pen combines a pregnancy test function into a light-weight, highly portable, airtight pen-like structure having self-contained drying agents to solve the defects of the prior art and provide a portable and high privacy pregnancy test instrument.

Those skilled in the art will readily observe that numerous modifications and alterations of the device may be made while retaining the teachings of the invention. Accordingly, the above disclosure should be construed as limited only by the metes and bounds of the appended claims.

What is claimed is:

1. A test pen comprising:
an integral hollow pen barrel divided into four sections in order being a dip section, a testing and observation section, a drying section, and a sealed rear section; wherein the dip section includes a front opening with a diameter allowing a slender water-absorbing member to pass therethrough, an air balance hole to facilitate liquid siphoning wherein the rear part of the slender water-absorbing member is cut apart to form two clips;
a pen cap comprising a hollow main body that is capable of capping the dip section, a flexible inner fringe for keeping the dip section in an airtight status, and an extending clipping memberfor clipping the test pen to a breast pocket;
a rear plug for sealing the rear section; and
a testing member, which is fastened by the two clips, including a support card, a solid carrier, a conjugate release pad partially overlapping a front end of the solid carrier, an absorbing pad partially overlapping a rear end of the solid carrier, and a cover tape.

2. The test pen of claim 1 wherein the drying section is loaded with at least a drying agent and is partitioned from the remainder of the integral hollow pen barrel by an extending fastener, wherein the extending fastener is an integral part of the integral hollow pen barrel between the drying section and the testing and observation section and separates the drying agents from the testing section.

3. The test pen of claim 1 wherein the testing member further includes a specimen-absorbing pad partially overlapping a front end of the conjugate release pad.

4. The test pen of claim 1 wherein the solid carrier is a rectangular nitrocellulose plate that is conducive to fluid movement and provides antigen-antibody bonding, specific areas of the solid carrier being covered with a binder to provide colorful testing results after reaction with a liquid specimen.

5. The test pen of claim 1 wherein the water-absorbing member extends through the front opening.

6. The test pen of claim 5 wherein the water-absorbing member comprises polyester fiber, glass fiber, or other water-absorbing material.

7. The test penof claim 1 wherein the conjugate release pad is composed of a low-density glass fiber.

8. The test pen of claim 1 wherein the integral hollow pen barrel includes a plurality of first slender protrusions for fixing the solid water-absorbing member, and a plurality of second slender protrusions for fixing testing reagents.
